(19) United States
(12) Patent Application Publication
(10) Pub. No.: US 2004/0091432 A1
(43) Pub. Date: May 13, 2004

(54) Oral hygiene system and method of treatment

(76) Inventor: Jacques M. Dulin, Sequim, WA (US)

Correspondence Address: Jacques M. Dulin, Esq., Innovation Law Group, Ltd., NetPort Center, Suite 201, 224 W. Washington St., Sequim, WA, 98382-3338, US

(21) Appl. No.: 10/700,784

(22) Filed: Nov. 3, 2003

Related U.S. Application Data
(60) Provisional application No. 60/423,539, filed on Nov. 4, 2002.

(51) Int. Cl. A61K 7/16; A61K 9/70

(52) U.S. Cl. 424/49; 424/443

(57) ABSTRACT

Portable medication delivery system for treatment of bad breath, gingivitis, ANUG, plaque, etc., comprising small, shirt pocket-sized re-closable pouches having 2 to 4 sterile, single-use, disposable, treated cotton rolls carrying a selected periodontal anti-microbial, such as mouthwash, or a fluoride, enzyme or antibiotic composition. The anti-microbial is preferably a neutral, alcohol-free solution, but may be a dry or gel composition. The treated rolls are placed in the upper and/or lower buccal vestibules where they release, over time, the anti-microbial solution. The treatment directly targets the critical areas, tiny “sulcus” grooves and spaces between the teeth, both significant reservoirs for bacteria. The rolls are left in place for extended periods, on the order of 5-30 minutes or more, without discomfort and without interfering with normal activities of speaking or travel. Tests show the inventive medicated rolls are truly effective in treatment of bad breath.

### CROSS-REFERENCE TO RELATED CASE

This is the Regular US Application and/or its PCT counterpart of U.S. Provisional Application S. No. 60/423,539, filed 4 November, 2002, by the inventor, entitled “Oral Hygene System, Dosage Units and Method”, the priority of which is claimed under 35 US Code 119, and related treaties.

### SPECIFICATION

1. Field

The invention relates to improvements in oral hygiene by use of portable, single-use, disposable dosage units for treatment of oral conditions of bad breath, gingivitis and plaque comprising small, shirt pocket-sized re-closable pouches containing from 2 to 4 or more sterile cotton rolls moistened or saturated with a medication, typically a periodontal antiseptic (anti-microbial) solution, such as a gel or solution of mouthwash or other periodontal solution and/or suitable antibiotic.

2. Background

“Bad breath” or “halitosis” refers to a wide range of oral conditions, from situational bad breath (“coffee breath”) to chronic periodontal-disease-related fetid breath. The current approach to treating bad breath is use of liquid mouth washes (mouth rinses), commercially available without prescription, for temporary breath freshening.

There are two major, dominant types of liquid mouthwashes: Boric Acid-types, such as Listerene® (a Pfizer brand) or its generic equivalents; and Benzoic Acid-types, such as Scope® (a Proctor & Gamble brand) or its generic equivalents. Boric acid-type includes boric acid, thymol, eucalyptol, ethanol and various colorants, flavoring, carriers, sweeteners and preserveatives. The boric acid, thymol and eucalyptol provide the “burn” and relatively unpleasant taste. The benzoic acid type has become a strong competitor to the boric acid type mouthwash because it has a mild to non-existent “burn” and is more palatable.

However, popular liquid mouthwashes primarily focus on flavor. In fact, the short “rinse” time of popular liquid mouthwashes, on the order of 10-30 sec-onds, is largely ineffective for more than a few minutes. Further, consumers in general do not know or realize that popular liquid mouthwashes are relatively acidic, in a range below pH 5.5. Demineralization of tooth enamel is known to occur below that pH, see Pontefract, H, et al, “The Erosive effects of Some Mouthrinses on Enamel. A Study in situ” J. Clinical Periodontology, Vol 28, No. 4, 391-324 (April 2001). For example, pHs of popular mouth rinses are 4.3 (Listerene®) and 5.4 (Scope®). In addition, popular liquid mouthwashes contain from about 14-28% ethyl alcohol (Scope®: 14-16%; Listerene®: 26-28%), and therefore are required to have child-proof caps to prevent alcohol toxicity in children.

Bad-breath-causing bacteria flourish on the tongue, between the teeth and in the sulcus grove (periodontal pocket) between the teeth roots and the gums. Flossing, either with filament, proxy brushes or water jets, is intended to clean between the teeth, while tongue brushes are for cleaning the tongue. However, such physical actions are only partly successful, and residual bacterial pockets remain. Further, such actions cannot be taken in public or on the move. One reality is that bad breath may surface during the work day, say after a break or lunch. Listerene® “Gel Strips”, breath freshener gum (Eclipse® of Wrigley), concentrated flavor drops (Binaca®), and “candy”-type dental lozenges (Tic-Tacs®, or Aqua-fresh® lozenges of Smith-Kline-Beecham) are targeted to that reality. However, those are either highly flavored bad breath cover-ups or are designed to simply stimulate saliva flow to flush the oral cavity. Further, they are not targeted to where treatment is needed most.

Thus, there is a real need in the field for a truly effective oral hygiene delivery system and method of treatment of offensive oral conditions such as bad breath, which system is simple, inexpensive, highly portable, can be used at any time, targets the locus of need for the medication, provides extended delivery periods and does not interfere with or restrict ordinary life activities. The invention meets those needs.

### THE INVENTION

### SUMMARY, INCLUDING OBJECTS AND ADVANTAGES

The invention is directed to a new form of oral hygiene product for the consumer market that provides significant advantages over current popular liquid mouthwashes. The inventive system and method comprises a single-use, disposable, moist, treated cotton roll insertable between the teeth and cheek with the tongue or fingers. It delivers a medicated solution, slowly and directly into the critical target areas, the tiny “sulcus” and the spaces between the teeth, both significant reservoirs for bad breath-causing bacteria. The inventive medicated rolls may be left in place for extended periods, on the order of 5-30 minutes or more, without discomfort and without interfering with normal activities of speaking or moving around. It is preferred to employ an alcohol-free anti-microbial solution that has a pH above about 5.5, preferably in the range of from about 5.5 to about 8.5.

The inventive medication-moistened or saturated treatment rolls are a new “platform” for delivery of topical oral medications for a wide range of periodontal conditions, such as bad breath (halitosis), gingivitis, ANUG and dry mouth syndrome. The targeted location and extended treatment period made possible by the inventive treated roll delivery system permits the medication to be more effective. The treated cotton rolls also provide a gum massaging effect upon the action of the buccinator muscle, which opens the free margin sulcus recess for access by the roll medication. This produces saliva, well recognized to be an effective, natural anti-bacterial.

The targeting provided by the inventive system is important in that less is more. That is, less anti-microbial applied at the locus of need is far more effective than a mouthful of mouthwash that is used for so short a period, seconds, as to be essentially ineffective for any substantial length of time. An important advantage of the inventive system is that the user can be active and talk while the treatment composition is applied to the gums. The user can insert rolls on the way to a meeting, say a 5 to 20 or more minute travel time, get on public transport, talk on a cell phone along the way and upon arrival simply dispose of the used rolls (back in the re-sealable pouch) just as he/she would do with chewing gum.

The inventive oral anti-microbial and drug delivery system is both a method and device for relatively slow release of treatment (medicated/antimicrobial) solutions or gels into the oral cavity. In a primary example, the device comprises a cotton roll, on the order of 3/16″ to 7/16″ in diameter by 1″-2″ long that is moistened or saturated with a treatment composition, e.g., an anti-microbial solution, for amelioration of bad breath, gingivitis, ANUG (Acute Necrotic Ulcerative Gingivitis), chronic periodontal disease, and other oral disorders. In a broader application, the rolls may be treated with a wide range of drugs and analgesics for delivery to the buccal mucosa and the gums. Examples include: For the former, use of antibiotics to assist in treatment of oral infections and diabetes; for the latter, saturation with a topical anesthetic such as xylocaine, benzocaine or euginol for treatment of teething in infants and for tooth aches until a dentist can be seen, or after oral surgery.

The act of using a mouthwash laves the tongue and lower jaw more than the upper, where contact is a matter of only a few seconds. In a substantial number of bad breath cases the source or reservoir is the sulcus in the upper jaw, not the lower. In contrast, the inventive medicated rolls can be placed in the upper buccal vestibules from which the treatment fluid medication is gradually released directly to the upper sulcus and between the upper teeth, and then descends to lave the tongue and lower jaw. The medicated rolls typically remain in the mouth for 10-20 minutes before they are depleted of medicated solution.

It should be clear that conventional mouthwashes can be used in the inventive delivery platform as the treatment solution. Surprisingly, in actual tests, delivery of boric acid mouthwash to the buccal vestibule by the treatment rolls does not result in the typical burning. That is, the boric acid mouthwash can be delivered by the treatment rolls to the buccal vestibules, both upper and lower for over 20 minutes without the usual burning sensation.

The preferred roll material is natural cellulose, cotton, sterilized before packing. In the adult version, the cotton rolls are “open ended”. That is, they have a thin cotton sheath or filamentary braiding around the circumference, but none at the ends, as they are cut to length from a longer piece of cotton roving. In the alternative, a soluble cellulose, such as oxycellulose or some easily biodegradable or enzymatically degradable polymer can be used. Likewise, a “mechanical” (structural) redesign of the rolls into a plurality of small “pellets” that are encased in a porous sheath can be used. In the unlikely event such a roll is swallowed, the polymer sheath would be degraded by a gut enzyme, not an enzyme present in saliva, thus providing that the sheath breaks apart in the gut and the pellets would be easily passed through the intestines. In regards to infants, an alternative design can employ a pacifier having a roll on a short tether. The roll is saturated with topical anesthetic or euginol to numb the gum area irritated by eruption of a tooth during teething. The child chews on the roll, or it is placed in the vestibule next to the inflamed gum area. In this instance, the roll would be more durable, and totally encaseed at the ends.

The inventive oral medication delivery system includes packaging a plurality of treatment rolls in plasticized paper or foil pouches, each on the order of 1.75″ to 2.25″ wide by 2″-3″ long with a re-sealable closure and a tear-off strip. From 2-4 or more rolls are packed per pouch and the dosage per roll can range from about 1-3 ml of treatment solution. The inventtive delivery system can be easily adapted to a wide range of product types, such as: 1) Consumer oriented medicated solution-containing rolls targeting the halitosis (bad breath) market; 2) Consumer or prescription Special Condition Medicated Rolls, targeting the market for special periodontal conditions, disease and procedures, such as: Dry Mouth; Pre- and Post-Op Oral Surgery treatment; ANUG; and 3) Consumer or Prescription Caries-Control Medicated Rolls, targeting the children and adolescent market. As to a delivery system for product types 1 and 2 above, the treated cotton rolls also can be used to carry a wide variety of drugs and over the counter medications, e.g.: topical antibiotic-containing oral solutions (for treatment of the cascade of severe periodontal infection accompanying Type 2 diabetes); chlorhexidene; quinones; aloe vera extracts or gels; enzymes; zinc compounds (for treatment of colds and bad breath from related post-nasal drip); fluoride compounds such as stannous fluoride (in limited concentrations below about 1.2 ppm) to inhibit plaque formation and associated caries and gingivitis, to treat xerostomia (dry mouth) and to enhance remineralization of tooth enamel; anti-inflammatories; analgesics; and anti-caries medications, to name a few. Other applications include use to prevent or reduce: supra-gingival plaque accumulation and associated caries, gingivitis and gingival inflammation: periodontitis; periodontal disease; canker sores, herpes labialis (Type 1 cold sores/fever blisters). The permeation characteristics of the vestibular mucosa, both buccal and gum mucosa, are unique, and treatment gels and solutions can be tailored to take advantage of those unique characteristics.

The presently preferred form factor for the inventive product system is to package four medication-containing rolls per shirt-pocket sized re-sealable pouch, in boxes of 30-60 pouches (1-2 months supply). The pouches are easily produced at high speed in food grade or cosmetics grade (sterile) packaging. The pouches are standard, commercially available bags (2″ wide×2-2.5″ long, with or without a gusset) that can be made to order on conventional resealable zip-type bag-making machines. The 4 cotton rolls are inserted into the open pouches oriented with the tear seal and the zip-type re-closeable seal down (i.e., fed into the open bottom). Then the selected aliquot amount of medication or treatment solution or gel is dispensed onto the rolls in the pouch, and the bottom edge heat-sealed. The top tear seal has an easy, manual tear-open notch feature. The product pouches are “up-scale”, attractive and distinctive in appearance, with corresponding box-packing in a 1-2 month-supply size. Point of sale countertop dispensers for sale of “singles”, and punch-holes in the top margin for “hanger” sales are other packaging alternatives.

Preferably inert gas, such as N₂ is used to displace air in the pouch during filling, and the excess N₂ is pressed out of the pouch at the point of heat sealing the bottom by passing between a pair of spaced rollers. The cotton rolls are highly absorbent and the amount of the treatment composition is selected and controlled to insure there is no excess fluid or gel in the pouches so that the bottom surfaces of the pouch to remain dry for sealing.

The consumer tears off the top seal, pulls open the zip seal, removes one or more rolls, places them on his/her tongue, and easily pushes them into the buccal vestibules. The user can speak on the phone, continue dressing, carry on a conversation, walk, sleep and even eat with the rolls in place for 30 minutes to an hour or more without discomfort. The buccinator muscles flatten the rolls to a comfortable oval shape, and in most instances, an observer is unaware the user has rolls in place.

A particularly useful embodiment of the inventive delivery system employs more neutral pH solutions, in the range from about 5.5 to about 8.5, to counter-act the erosive effects of low pH mouthwashes. Calculus (tartar) is formed when saliva enters the oral cavity that has been acidified by soda pop, sweets and popular liquid mouthwashes and the calcium and phosphate ions in the saliva precipitate out in the acid conditions. This promotes calcification of the existing plaque. With the higher pH solutions, calculus formation is inhibited. In addition, the massaging effect of the presence of the cotton rolls stimulates added flow of saliva to help flush soda pop and plaque acids.

### BRIEF DESCRIPTION OF DRAWINGS

The invention is shown in more detail by reference to the drawings in which:

FIG. 1 is an isometric view of the portable, pocket sized, single or double serve pouch with a tear-off top and optional resealable press-lock beading (Zip-Lock® type reclosable seal strip) or reclosable adhesive seal strip, and having 4 anti-microbial-saturated cotton rolls packaged therein;

FIG. 2 is an isometric view of individual treatment rolls, in which FIG. 2A shows a roll having a cotton fiber core encased in an external woven or non-woven cotton or paper wrapper, and FIG. 2B shows a cotton roll in which the fibers are retained in roll shape with fine plastic filament braided around the cotton to retain in roll form;

FIG. 3 is an isometric view of an individual carry pouch showing a first roll removed after the seal strip is torn off along perforation lines, and the inner zip-type seal strip opened;

FIG. 4 is a simplified vertical section view through a patient's or user's mouth showing the placement of the treatment rolls in place in the upper and lower buccal vestibules;

FIG. 5 is an isometric view of a consumer package comprising a box containing a plurality of pouches; and

FIG. 6 is a flow sheet of one exemplary method of preparing the pouches and packaging them in product boxes for consumer distribution.

### DETAILED DESCRIPTION, INCLUDING THE BEST MODE OF CARRYING OUT THE INVENTION

The following detailed description is by way of example and not by way of limitation of the scope and principles of the inventive method and apparatus for treatment of adverse oral conditions, the dosage units and the system of packaging. One skilled in this art will readily understand the invention and will be able, without undue experimentation, to apply the inventive treatment system to treat a wide variety of conditions and to apply other conventional packaging models and modalities to design a wide variety of equivalent and alternative packaging systems for the inventive dosage units.

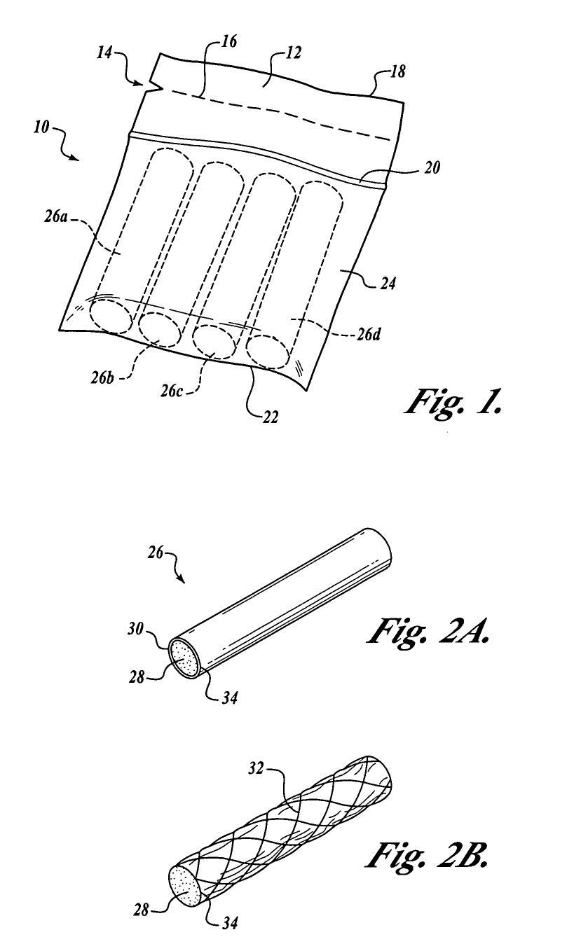

FIG. 1 shows the inventive portable, shirt pocket-sized packet 10 which comprises a water impervious pouch containing from 2 to 4 or more antimicrobial solution or gell-saturated, single-use cotton rolls 26a-26d in the product compartment 24. The pouch includes a marginal strip 12 that is torn off to reveal the inner re-closable seal strip assembly 20. The tear-off strip removal is initiated using the starter notch 14 and a micro-perforated or other type of release line 16. The release line may also be a line of weakening, or a foil, string or other type release pull. The re-closable assembly may be any suitable closure, such as a Zip-Lock® type inter-fitting bead and groove construction, a strip of re-sealable pressure-type adhesive, or the like.

The pouch is preferably a plastic, metallized plastic or laminated paper pouch, the latter comprising a paper outer layer with a plastic inner layer. Any suitable water and alcohol impervious material may be used, but the exterior surface of the pouch material needs to be printable to carry the product name and usage instructions. In a simple form, a 4-mil printable polyethylene can be used to form the pouch 10, but a dual- or multi-layer water and air impervious packaging plastic, metallized plastic, or plastic coated or laminated paper can be used. Typical film thicknesses range from 1-5 mils.

One skilled in the packaging art can select the pouch material to suit the desired properties: shelf life; sanitation, clarity; mechanical strength; trapped print (print on one layer laminated to a clear-over layer); sealability; permeability; etc. A suitable supported film can be made of nylon or PET laminate, or a PET-supported polyolefin (e.g., PE, polyethylene, or PP, polypropylene) film, or a PET/Saran/LDPE or HDPE film (PET is a polyethylene terephthalate and Mylar® is a commercially available metallized PET film made by duPont that is particularly attractive for packaging). To package the rolls 26 in the pouches 10, the pouch is formed with the bottom open and inverted, the rolls inserted, the medicated solution aliquot introduced and the bottom then sealed along line 22.

The pouches of this invention are approximately 2″×2.5″, as the cotton rolls 26a-26d are each approximately 1.5″ long by ¼″ to ⅜″ in diameter. Roll material should be medical quality and purity, sterile and have excellent, rapid absorbency and retention of the selected treatment fluid or gel. Multiple pouches are conveniently packaged in a small box, such as 10-30 packets or pouches in a box approximately 2¼″ to 2 1/2″ wide, by the same high, by 4½″ to 7″ long (approximately the size of a box of tea bags). Other packaging configurations are entirely feasible, such as boxes the approximate size and configuration of anti-histamine or throat lozenge packaging. Alternately, the pouches can be packaged in plastic bags with hanger holes or header cards with hanger holes. The pouches can be attached to each other in a strip with perforated separator lines between them, and can be packed in a fan-folded configuration.

FIG. 2 shows two embodiments of the cotton rolls 26 useful in this invention. FIG. 2A shows a roll of cotton fibers 28 encased in a woven or non-woven cotton, paper or polyester/rayon fabric sheeting 30. Alternately, the outer layer may be any micro-perforated plastic sheeting. FIG. 2B shows the presently preferred embodiment, a 5/16″ diameter roll of 1.5″ length of cotton fibers encased or retained in a fine cotton or plastic filament 32 in a braided or interlaced configuration. This preferred embodiment is called “braided roll” and employs a nylon and acetate braiding filament. Both roll embodiments are commercially available from dental supply firms.

Although pure natural cotton is presently preferred for rolls, any suitable polymer in fiber or rolled-up sheet form that is capable of absorbing and releasably retaining treatment solution or gel may be used. For example, cotton and some polymers can be chemically treated to include hydrogen bonded or covalently bonded groups that have anti-bacterial or anti-pathogenic activity. Due to the outer layer 30 or the braiding filament 32, the rolls retain their integrity until and during use and for disposal. After use, the rolls can be removed from the mouth without threads or filaments of cotton remaining in the oral cavity or snagged on teeth, braces, fillings or other dental appliances.

As shown in FIG. 2 the ends of the roll 26 are transversely cut 34, with the core exposed. However, the sheeting 30 or braiding 32 may be folded or gathered over the ends and sealed to totally encapsulate the absorbent core material. A less expensive variation would be to omit the outer layer, but while such a pure cylindrical (or other geometric prism-shaped form) of cotton roll or batting without a covering can be used, it is not preferred due to the shedding of cotton strands and filaments. The medicated solution in gel form can assist in retaining the integrity of the roll, but in actual practice the covering 30 or braiding 32 of FIG. 2B is preferred.

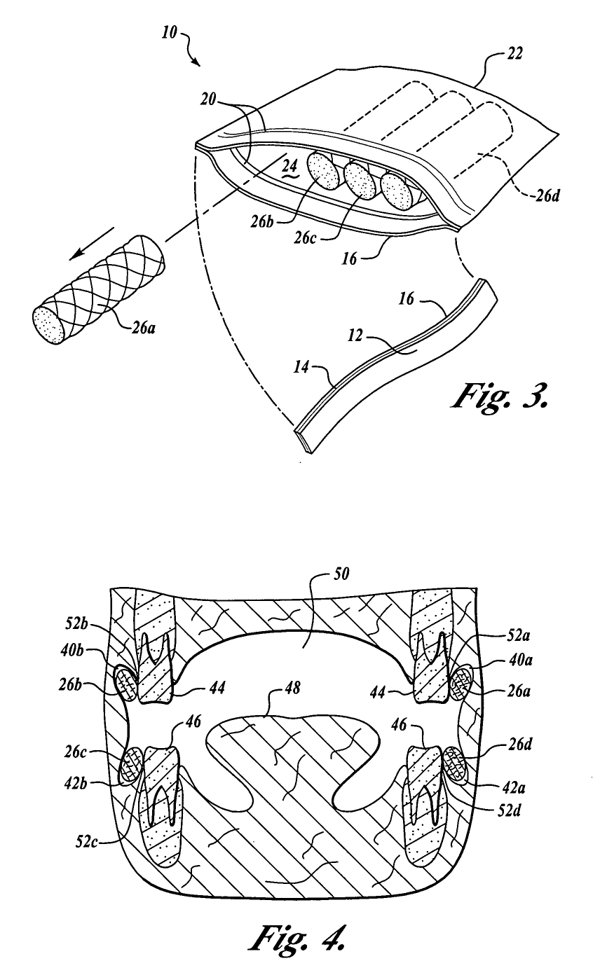

FIG. 3 is an isometric view of a pouch 10 of the invention in use. The strip 12 is removed by tearing along the line 16 to reveal the re-seal assembly 20 which is opened to access the four moistened or saturated braided rolls 26a-26d. If only two rolls are needed, then two are removed and inserted, and the pouch resealed by pressing the seal strip 20 bead into the receiving channel. The pouch may then be placed back into a purse or pocket, such as a shirt or coat pocket until the next use is desired. Used rolls can be re-sealed in the pouch and disposed when a trash receptacle is at hand.

FIG. 4 shows the location of placement of the inventive treatment rolls 26a-26d. Rolls 26a and 26b are placed in the upper buccal vestibules 40a and 40b adjacent the upper molars 44, and saturated treatment rolls 26c and 26d are placed in the lower vestibules 42a and 42b, respectively, adjacent the lower molars 46. Upon gentle squeeze of the cheeks, the treatment solution or gel is released from the rolls and the solution laves the adjacent gums, sulcus and teeth, exactly where it is needed most. In addition, the lateral pressure of the cheeks forces solution between the teeth. Enough solution is released to migrate into the oral cavity 50 where it bathes the cavity and tongue 28. The massaging action of the buccinators opens the sulcus 52 and the solution penetrates into those pockets.

With respect to the solutions or gels, both benzoic acid (Scope®-type) and boric acid (Listerene®-type) mouthwashes can be used. Suitable roll medications in fluid, gel or dry form can include one or more of the following as active ingredients: thymol; eucalyptol; aloe vera; methyl salicylate; menthol; alcohol (ethyl); benzoic acid; sodium benzoate; boric acid; citric acid; calcium lactate; cetyl pyridinium chloride; tetrasodium pyrophosphate; benzalkonium chloride; domiphen bromide; sodium benzoate; euginol; eucalyptus oil; oil of cloves; triclosan; chlorhexidine and chlorhexidine gluconate; sodium hypochlorite; sodium stannate; hydrogen peroxide; carbamide peroxide; garlic formulations (e.g., Garlique®); antibiotics; zinc-containing compounds and complexes such as zinc gluconate or zinc chloride; chlorine dioxide; fluoride ion-providing compounds such as sodium fluoride; enzymes and enzymatic compounds; copper gluconate; and other suitable anti-bacterials; and oral cavity and throat treatment compounds and formulations. Examples of useful enzyme-type anti-bacterial systems can include one or more of lysozyme, lactoferrin, glucose oxidase, lactoperoxidase, and the like. Analgesic compounds may also be used in the medication composition, including: aspirin, methyl salicylate; acetomenophen; naproxen sodium and the like; topical anesthetics such as xylocaine, benzocaine, euginol, and the like. Likewise, a wide range of natural plant herb, oil or extracts having anti-microbial or other therapeutic or ameliorating properties can be used as the effective ingredients in appropriate dosages, such as: aloe vera, resveratrol, naphthoquinones, juglone, 7-methyljuglone, psylocarpene, and the like, The effective ingredients can be in any form: in carriers, in gels, and dry. Effective weight, volume or mole percentages of the effective ingredients are well established, or easily can be determined by those skilled in the art.

In addition, suitable carriers, colorants, flavorings and carriers, such as, but not limited to: water; de-ionized water; poloxamer 407; polysorbate 20 and 80; Tween 20; potassium sorbate; dibasic sodium phosphates; sodium benzoate; sodium chlorate; sodium citrate; sodium lauryl sulfate; PEG 40; hydrogenated castor oil; silicone emulsion; citric acid; edentate calcium disodium; menthol; cocomidopropyl betaine; sodium saccharin; isomalt; mannitol; lactitol; acesulfame-K; magnesium stearate; sorbitol; xylitol; glycerin; sugars; saccharine; propylene glycol; carboxymethyl cellulose; hydroxyethyl cellulose; calcium lactate; aloe vera; natural flavors such as mint, peppermint, cinnamon and clove; hydrogenated starch hydrolysate; thickeners such as xanthan gum; and the like, can be used for distinctive, pleasant or ameliorative flavorings, texturants, mouth feel (organoleptic) properties and colors. In addition, the packaging or/and the rolls or solutions/gels used can be color-coded to identify different ingredient mixes, strength levels, and types or frequency of usage.

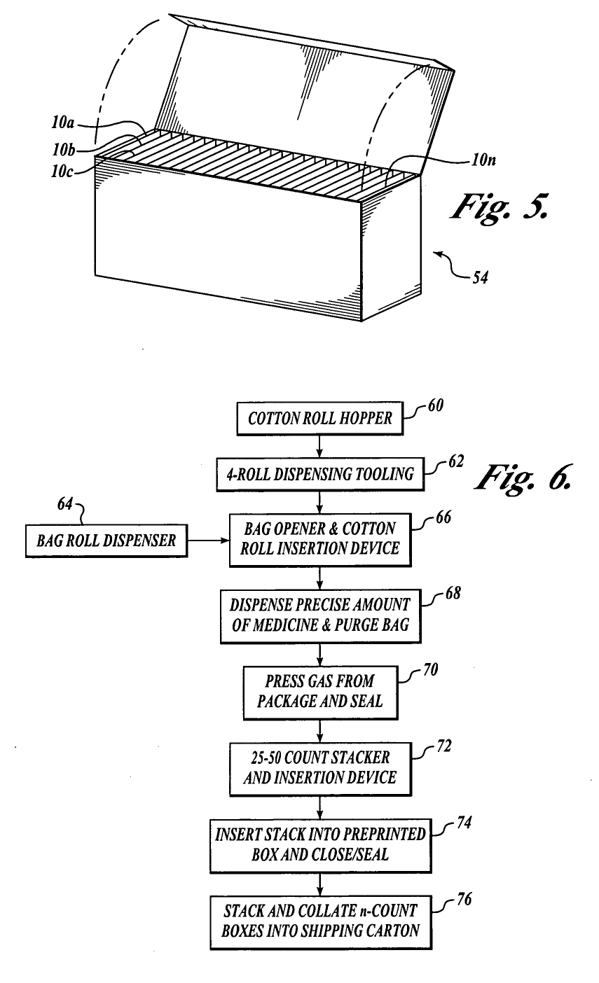

FIG. 5 shows a typical consumer package, a box 54 containing from 10-50 pouches, 10, typically 20-30 pouches. The box can be over-wrapped in clear plastic or cellophane. For “deal”, bulk or warehouse store packaging 2 or more boxes can be co-wrapped.

FIG. 6 is a flow sheet of one exemplary packaging apparatus system and process for packing the rolls in the pouches and the pouches in the consumer distribution boxing. Station 60 comprises a cotton roll feed hopper into which are placed the sterile, dry cotton rolls. The hopper may be similar to a cigarette filling machine. The cotton rolls are placed with their axes normal to the hopper sides and they “drain” via a sloped bottom to the dispensing area. The hopper may include a timed rotary incrementing metering wheel having fingers that maintain groups of 4 rolls in alignment and adjacent to each other. A slight upward air stream may be used to keep the individual rolls or groups of 4 rolls separate. In the alternative, a vacuum feeder, such as a Coanda-type induced vacuum lifter can pick individual rolls, or groups of 2-4 rolls at a time, from a conveyor belt or other feeder and forward them to the dispensing tooling station 62.

Dispensing station 62 comprises tooling to hold the 4 rolls in position for inserting them into an open pouch (see station/step 66 below). By way of example, this tooling may “cluster” the four rolls, bringing them into parallel axial alignment to form a diamond-shaped or generally circular-shaped cross-section to provide a smaller size (cross-section) for “stuffing” into the pouch. Alternately, as the rolls are somewhat compressible, they can be aligned 4-abreast and then squeezed closer together for insertion into the pouch. An appropriately-shaped funnel feeder and pusher mechanism can be easily provided by those skilled in the art of packaging and materials handling.

At the same time, continuous, pre-formed and pre-printed pouches are supplied on a spool at bag roll station 64. The pouches are fabricated with a “zip” lock feature with a preformed tear-off seal above the zip-lock (as shown in FIGS. 1 and 3). For this example, each pouch is 2″ wide and about 2⅜″ long, with the extra length allowing about ½″ for the zip-lock feature (20 in FIG. 1) and the space to the upper seal (12, 14 and 16 in FIG. 1). Also, about ⅛″ is to allow space for the bottom seal which occurs at station 70. The bottom of the pouch (22 in FIG. 1) is open (unsealed), and the pouches are provided upside down to the filling station 66. The bag material is preferably a supported plastic film having an exterior printable surface, the plastic being suitably impervious to oxygen and CO₂ intake, and water vapor, alcohol and N₂ out-gassing, The continuous strip of pouches dispensed from station 64 is fed from a spool mounted on a back-tensioning shaft to the pouch filling station 66.

At the filling station 66, the end of the strip of pouches are held firmly by movable fingers or opposed compressing rollers or tractor drives to move the pouches through the filling station. At this station, the bottom of the pouch is opened either by one or more of mechanical means, pneumatic means (an air or N₂ jet), or by use of vacuum cup or vacuum suction devices. Once opened, the cotton roll dispensing tooling of station 62 inserts groups of 4 rolls into the open end of the pouch and a push arm holds the rolls in place as the tooling is retracted. This push bar can also maintain the pouch open for the next step of dispensing the medication aliquot onto the rolls in the pouch in dispensing station 68.

An automatic, adjustable dispenser in station 68 is used to add a specified amount of medication, e.g., 6-10 ml aliquot of antimicrobial in the case of a bad breath product, to the rolls in each pouch. The aliquot can be a single 4-12 ml aliquot delivered generally to all rolls simultaneously, or individual aliquots of 0.75-3 mls may be directed to the axial center of each roll at its exposed end. This measured amount of treatment solution is rapidly absorbed into the cotton rolls and retained there by capillary action. A small amount of N₂ is directed into the open pouch to displace the air and provide an inert atmosphere within the pouch.

The filled pouches proceed to sealing station 70, where they are fed or are drawn between rollers or tractor drive that compresses the pouch sides to express excess N₂ and bring the up-raised bottom ¼″ or so of the pouch sides together for sealing. The compression is controlled adjacent the area of the rolls to insure the aliquot of fluid is not forced up into the seal area. Stations 68 and 70 may be combined into one station. Once the pouch is sealed in station 70, a sharp blade cuts the pouch from the continuous roll just above the top seal, and the now-filled and sealed individual bags are conveyed to stacking station 72.

At stacking station 72, the conveyed pouches are collected into groups appropriate for the size of the consumer box (54 in FIG. 5) and forwarded to box filling station 74 where they are inserted into the box, the box closed and sealed, or over-wrapped with clear plastic or cellophane. At station 76 the boxes are stacked, collated and packed into shipping cartons. The box packing stations 74-76 can be combined into a single station, or 72 and 74 into a single station and the packing at station 76 done automatically or by hand.

In an alternate packaging method, the rolls can be moistened or saturated with the selected treatment solution before being introduced into the pouches.

### INDUSTRIAL APPLICABILITY

The liquid mouthwash market is world wide, ranging from age 15 to 80+. The market, in excess of $1 Billion annual US sales, is growing at the rate of 3-4% annually. By comparison to commercially available mouthwashes, the inventive treated cotton rolls provide directly targeted, and longer term delivery of anti-microbial solution where it is needed most. The inventive delivery system has real potential to significantly improve periodontal health, both for children and for adults with persistent bad breath, gingivitis, ANUG, and similar periodontal diseases. A wide variety of proprietary oral hygiene solutions can be used; of particular usefulness are those having non-enamel-etching pH, low or no alcohol and better anti-microbial efficacy.

Normal good dental hygiene practices of regular brushing and flossing should not be eliminated. But at the first sign of bad breath the inventive medicated rolls can be effective.

Other advantages of the invention include:

More targeted, concentrated application directly at the point of need;

Longer application of proper dosage amounts for greater effectiveness;

Portable, light weight, non-breakable, non-spillable, disposable packaging;

The user can be fully active during treatment;

It is discreet and particularly useful during travel, long meetings, or other occasions when users cannot floss, brush or gargle and at events that include food/beverages causing quick development of bad breath;

By squeezing the cheeks, the fluid or gel is forced into oral cavity and tongue for a more gentle, metered, longer treatment without burning;

Permits use of stronger formulations that otherwise might generate discomfort or burning of the tongue;

The solutions can be selected for the treatment desired: bad breath, or antibiotics and throat care compositions for symptoms of colds, sore throats, etc; and

The pouches are easy to manufacture and fill, being well within the scope of available packaging technology to print, manufacture, fill and package in boxes or bags for ease of distribution.

Those skilled in the art will recognize that the invention, in its various aspects, provides a particularly useful consumer product. Indeed, any of the commercially available mouthwashes, mouth rinses and oral hygiene product solutions can be used as medications for the cotton roll delivery system of this invention, including, without limitation: Listerene® and Store Brand Equivalents; Scope® and Store Brand Equivalents; Act® (Johnson & Johnson); Plax(D (Pfizer); Breath Remedy® (US DenTek, Corp.); Biotene® antibacterial enzyme system (Laclede, Inc.); Prevention™ periodontal & orthodontic mouth rinse; Advanced Breath Care® (Arm & Hammer); Cepacol®; Targon®; Peridex®, Colgate Phosflur®; Colgate Peroxyl®; Florigard(D (Colgate); Therabreath®; Oragel Perioseptic®; BreathRx (Discus Dental, Inc); Peridex®; Periogard®, and the like.

The suggested dosage of popular liquid mouthwashes is 20 ml so that 1 Litre provides 50 applications and 1.5L provides 75 applications. In contrast, the inventive medicated roll system as a dosage unit need employ only from 0.75-3 ml, a total of 4-12 per application, some 40-80% less than popular liquid mouthwashes, but with equal or greater effectiveness.

It should be understood that various modifications within the scope of this invention can be made by one of ordinary skill in the art without departing from the spirit thereof and without undue experimentation. For example, the treatment composition or medication need not fully saturate the rolls; rather, they may be moistened (less than saturated), or the medication can be dry, in which case the mouth saliva provides a moistening and dissolving effect over time for more controlled or delayed release of the medication. This invention is therefore to be defined by the scope of the appended claims as broadly as the prior art will permit, and in view of the specification if need be, including a full range of current and future equivalents thereof.

1. A medication delivery system for oral hygiene comprising;
a) a cotton roll of size and length to be received comfortably in a buccal vestibule; and
b) a treatment composition carried by said roll in an amount to be therapeutically effective to treat adverse periodontal conditions.

2. A medication delivery system for oral hygiene as in claim 1 wherein said medication is in a state selected from a liquid, a gel and a dry state.

3. A medication delivery system for oral hygiene as in claim 2 wherein said composition is selected from an anti-microbial compound or composition, a fluoride-ion providing compound or composition, an analgesic, an enzyme, a natural plant herb, oil or extract, an anti-plaque compound or composition, and combinations thereof.

3. A medication delivery system for oral hygiene as in claim 3 wherein said composition is an anti-microbial present in said roll in an effective amount for amelioration of bad breath conditions.

4. A medication delivery system for oral hygiene as in claim 3 wherein said composition is a fluid and said fluid is present in an amount sufficient to substantially moisten or to saturate said cotton roll.

5. A medication delivery system for oral hygiene as in claim 3 wherein said composition includes at least one active ingredient selected from a boric acid-type and a benzoic acid-type mouthwash.

6. A medication delivery system for oral hygiene as in claim 3 wherein said composition includes at least one active ingredient selected from: thymol; eucalyptol; methyl salicylate; menthol; ethyl alcohol; benzoic acid; sodium benzoate; boric acid; citric acid; calcium lactate; cetyl pyridinium chloride; tetrasodium pyrophosphate; benzalkonium chloride; domiphen bromide; sodium benzoate; euginol; eucalyptus oil; aloe vera; oil of cloves; triclosan; chlorhexidine; chlorhexidine gluconate; sodium hypochlorite; sodium stannate; hydrogen peroxide; carbamide peroxide; anti-biotics; analgesics; chlorine dioxide; zinc-containing compounds and complexes such as zinc gluconate or zinc chloride; fluoride ion-providing compounds such as sodium fluoride; enzymes and enzymatic compounds selected from lysozyme, lactoferrin, glucose oxidase, lactoper-oxidase, and the like; copper gluconate; natural plant extracts having anti-microbial or therapeutic properties; and mixtures thereof.

7. A medication delivery system for oral hygiene as in claim 2 wherein said cotton roll has a diameter ranging from about 3/16″ to 7/16″ and length ranging from about 1″ to about 2″.

8. A medication delivery system for oral hygiene as in claim 7 wherein said cotton roll includes a core of absorbent cotton fibers and a sheath selected from a mesh braiding and a highly permeable woven or non-woven sheet material.

9. A medication delivery system for oral hygiene as in claim 8 wherein said cotton roll is a braided cotton roll having a diameter of about 5/16″ and length of about 1½″, and each end is terminated in a transverse cut.

10. A portable consumer package for oral hygiene treatment comprising in operative combination:
a) a sealed, water impervious re-closable pouch; and
b) a plurality of single use medication delivery units disposed in said pouch, each of which comprises:
i) a sterile cotton roll of size and length to be received comfortably in a buccal vestibule; and
ii) a composition carried by said roll in an amount to be therapeutically effective to treat adverse periodontal conditions.

11. A portable consumer package for oral hygiene treatment as in claim 10 wherein said pouch is pocket-sized and includes a manually openable seal member.

12. A portable consumer package for oral hygiene treatment as in claim 11 wherein said seal member is a tear off strip and a zip-type closure permits re-closing said pouch.

13. A portable consumer package for oral hygiene treatment as in claim 11 which contains from 2 to 4 of said cotton rolls and wherein said composition is selected from an antimicrobial compound or composition, a fluoride-ion providing compound or composition, an analgesic, an enzyme, a natural plant herb, oil or extract, an anti-plaque compound or composition, and combinations thereof.

14. A portable consumer package for oral hygiene treatment as in claim 13 wherein said composition is a fluid and said fluid is present in an amount sufficient to substantially moisten or to saturate said cotton roll, said pouch ranges in width from about 1.75″ to about 2.25″ and in length from about 2-2½″ and each said cotton rolls has a diameter ranging from about 3/16″ to 7/16″, a length ranging from about 1″ to about 2″, a core of absorbent cotton fibers and a sheath selected from a mesh braiding and a highly permeable woven or non-woven sheet material.

15. A portable consumer package for oral hygiene treatment as in claim 10 wherein a plurality of said pouches are packaged in a box.

16. A method of oral hygiene care comprising the steps of:
a) providing a single-use sterile cotton roll of size and length to be received comfortably in a buccal vestibule and having a treatment composition carried by said roll in an amount to be therapeutically effective to treat adverse periodontal conditions;
b) inserting a said treatment composition-carrying roll in a buccal vestibule for a time sufficient to provide some amelioration of said periodontal condition; and
c) removing and disposing of said single use cotton roll after sub-stantial leaching-out of said treatment composition to the oral tissue.

17. A method of oral hygiene treatment as in claim 16 wherein said composition is in a state selected from a liquid, a gel and a dry state.

18. A method of oral hygiene treatment as in claim 17 wherein said composition is selected from an anti-microbial compound or composition, a fluoride ion-providing compound or composition, an analgesic, an enzyme, a natural plant herb or extract, an anti-plaque compound or composition, and combinations thereof.

19. A method of oral hygiene treatment as in claim 18 said composition includes at least one active ingredient selected from: thymol; eucalyptol; aloe vera; methyl salicylate; menthol; ethyl alcohol; benzoic acid; sodium benzoate; boric acid; citric acid; calcium lactate; cetyl pyridinium chloride; tetrasodium pyrophosphate; benzalkonium chloride; domiphen bromide; sodium benzoate; euginol; eucalyptus oil; oil of cloves; triclosan; chlorhexidine; chlorhexidine gluconate; sodium hypochlorite; sodium stannate; hydrogen peroxide; carbamide peroxide; antibiotics; analgesics; chlorine dioxide; zinc-containing compounds and complexes such as zinc gluconate or zinc chloride; fluoride ion-providing compounds such as sodium fluoride; enzymes and enzymatic compounds selected from lysozyme, lactoferrin, glucose oxidase, lactoperoxidase, and the like; copper gluconate; natural plant extracts having anti-microbial or therapeutic properties; and mixtures thereof.

20. A method of oral hygiene treatment as in claim 16 wherein said composition is a fluid or gel present in an amount sufficient to substantially moisten or to saturate said cotton roll, and each said cotton roll has a diameter ranging from about 3/16″ to 7/16″, a length ranging from about 1″ to about 2″, a core of absorbent cotton fibers and a fiber-retaining sheath selected from a mesh braiding and a highly permeable woven or non-woven sheet material.
